(19) United States
(12) Patent Application Publication
(10) Pub. No.: US 2009/0170862 A1
(43) Pub. Date: Jul. 2, 2009

(54) USE OF C-KIT INHIBITORS FOR THE TREATMENT OF MYELOMA

(76) Inventor: Kenneth C. Anderson, Wellesley, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/393,418

(22) Filed: Feb. 26, 2009

Related U.S. Application Data
(63) Continuation of application No. 10/489,643, filed on Aug. 5, 2004.
(60) Provisional application No. 60/325,491, filed on Sep. 27, 2001.

(51) Int. Cl. A61K 31/501; A61K 31/496; A61P 35/00

(52) U.S. Cl. 514/252.02; 514/252.18

(57) ABSTRACT

The present invention relates to the use of a c-kit inhibitor, especially a c-kit inhibitor of formula I,

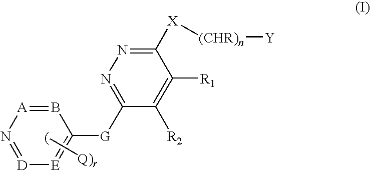

wherein the radicals and symbols have the meanings as defined in the specification, for the preparation of a medicament for the treatment of myeloma, in particular multiple myeloma, especially myeloma which is resistant to conventional chemotherapy; to a combination comprising a c-kit inhibitor and a compound effecting apoptosis of myeloma cells, preferably dexamethasone, for simultaneous, separate or sequential use; to methods of treating myeloma; and to a pharmaceutical composition comprising said combination.

The present invention relates to the use of a c-kit inhibitor for the preparation of a medicament for the treatment of myeloma; a method of treating a warm-blooded animal, especially a human, having myeloma, comprising administering to said animal a therapeutically effective amount of a c-kit inhibitor, especially a compound of formula I as defined herein; to a combination comprising a c-kit inhibitor and a compound effecting apoptosis of myeloma cells, preferably dexamethasone, and optionally at least one pharmaceutically acceptable carrier, for simultaneous, separate or sequential use; and to a pharmaceutical composition and a commercial package comprising said combination.

The term “myeloma” as used herein relates to a tumor composed of cells of the type normally found in the bone marrow. The term “multiple myeloma” as used herein means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia. Multiple myeloma is incurable by the use of conventional and high dose chemotherapies.

The compounds of formula I as defined herein and, in particular, PTK787 (also known as ZK222584) are tyrosine kinase inhibitors which were designed to inhibit the vascular endothelial growth factor (VEGF) signal transduction by binding directly to the ATP-binding sites of VEGFRs. The drug is most specific for KDR, but can also inhibit Flt-1 and Flt-4 and has activity against other tyrosine kinase receptors, including c-Kit. PTK787 inhibits the growth of several human carcinomas transplanted orthotopically into mice, including the A431 epidermoid carcinoma, Ls174T colon carcinoma, HT-29 colon carcinoma, and PC-3 prostate carcinoma as described by J. Wood, G. Bold, E. Buchdunger, et al. in Cancer Res. 60: 2178, 2000. PTK787 does not have a direct effect on any of these tumor cells, but does reduce vessel density in the tumor tissues, suggesting that its primary mode of action in these cells is through inhibition of angiogenesis.

Surprisingly, it was now found that c-kit inhibitors, especially the compounds of formula I as defined herein and, in particular, PTK787 directly inhibit the proliferation of myeloma cell lines and patient myeloma cells that express Flt-1. In addition, such compounds inhibit myeloma cell migration, assayed via transwell cell migration assay. Furthermore, such compounds, especially PTK787, can inhibit both proliferation of myeloma cells that are adherent to bone marrow stromal cells (BMSCs), and the secretion of IL-6 induced by binding of myeloma cells to BMSCs.

Hence, the invention relates to the use of a c-kit inhibitor for the preparation of a medicament for the treatment of myeloma, in particular of the use of a c-kit inhibitor of formula I

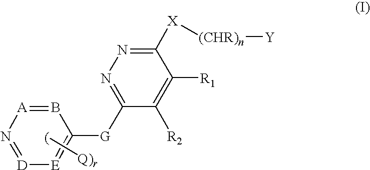

wherein
r is 0 to 2,
n is 0 to 2,
m is 0 to 4,
R₁ and R₂ (i) are lower alkyl or
(ii) together form a bridge in subformula I*

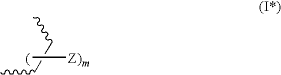

the binding being achieved via the two terminal carbon atoms, or
(iii) together form a bridge in subformula i**

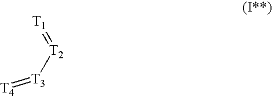

wherein one or two of the ring members T₁, T₂, T₃ and T₄ are nitrogen, and the others are in each case CH, and the binding is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom, or the salt of such compound having at least one salt-forming group.

The radicals and symbols as used in the definition of a compound of formula I have the meanings as disclosed in WO 98/35958 which publication is hereby incorporated into the present application by reference.

The present invention pertains in particular to the use of c-kit inhibitors for the preparation of a medicament for the treatment of myeloma, which is resistant to conventional chemotherapy.

The term “c-kit inhibitor” as used herein relates to a compound which shows activity, e.g., in the following c-kit enzyme assay:

The baculovirus donor vector pFbacG01 (GIBCO) is used to generate a recombinant baculovirus that expresses the amino acid region amino acids 544-976 of the cytoplasmic kinase domains of human c-Kit. The coding sequences for the cytoplasmic domain of c-Kit is amplified by PCR from a human uterus c-DNA library (Clontech). The amplified DNA fragment and the pFbacG01 vector are made compatible for ligation by digestion with BamH1 and EcoRI. Ligation of these DNA fragments results in the baculovirus donor plasmid c-Kit. The production of the viruses, the expression of proteins in Sf9 cells and the purification of the GST-fused proteins are performed as follows:

Production of virus: Transfer vector (pFbacG01-c-Kit) containing the c-Kit kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in 25 cm² flasks with the viral DNA using Cellfectin reagent.

Determination of small scale protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10⁷ cells/plate and infected with 1 mL of virus-containing media (approx. 5 MOIs). After 3 days the cells are scraped off the plate and centrifuged at 500 rpm for 5 min. Cell pellets from 10-20,100 cm² plates, are resuspended in 50 mL of ice-cold lysis buffer (25 mM Tris-HCl, pH 7.5, 2 mM EDTA, 1% NP-40, 1 mM DTT, 1 mM PMSF). The cells are stirred on ice for 15 min and then centrifuged at 5000 rpms for 20 min.

Purification of GST-tagged protein: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is eluted by 10 applications (1 mL each) of 25 mM Tris-HCl, pH 7.5, 10 mM reduced-glutathione, 100 mM NaCl, 1 mM DTT, 10% Glycerol and stored at −70° C.

Kinase assay: Tyrosine protein kinase assays with purified GST-c-Kit are carried out in a final volume of 30 μL containing 200-1800 ng of enzyme protein (depending on the specific activity), 20 mM Tris-HCl, pH 7.6, 3 mM MnCl₂, 3 mM MgCl₂, 1 mM DTT, 10 μM Na₃VO₄, 5 μg/mL poly(Glu,Tyr) 4:1, 1% DMSO, 1.0 μM ATP and 0.1 μCi [γ³³ P] ATP. The activity is assayed in the presence or absence of inhibitors, by measuring the incorporation of ³³P from [γ³³P] ATP into the poly(Glu,Tyr) substrate. The assay (30 μL) is carried out in 96-well plates at ambient temperature for 20 min under conditions described below and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 min with methanol rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄ and once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C.

A c-kit inhibitor as used for the present invention displays in the assay described above preferably an IC₅₀ value between 50 and 2500 nM, more preferably between 250 and 2000 nM, and most preferably between 500 and 1250 nM.

Furthermore, the invention relates to a method of treating myeloma, especially myeloma which is resistant to conventional chemotherapy, comprising administering a therapeutically effective amount of a c-kit inhibitor to a warm-blooded animal, in particular a human, in need thereof, preferably a therapeutically effective amount of a compound of formula I as defined above or an N-oxide of the defined compound, wherein 1 or more N atoms carry an oxygen atom, or the salt of such compound having at least one salt-forming group, to a warm-blooded animal, preferably a human, in need thereof.

The term “PTK787” as used herein means a compound of formula I wherein r, n and m are each 0, R₁ and R₂ together form a bridge of subformula I*, A, B, D and E are each CH, G is methylene, X is imino, Y is 4-chlorophenyl, and the bonds characterized by a wavy line are double bonds.

A preferred compound of formula I is PTK787. More preferably, PTK787 is employed in the form of its succinate salt.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

Throughout the present specification and claims myeloma means preferably multiple myeloma (MM).

The term “treatment” as used herein comprises the treatment of patients having myeloma or being in a pre-stage of said disease which effects the delay of progression of the disease in said patients.

Dexamethasone is a principle agent for the treatment of MM effecting apoptosis of myeloma cells. Surprisingly it was found that PTK787 adds to the effect of dexamethasone on MM cells. Furthermore, Interleukin-6 (IL-6) is a major growth and survival factor for MM cells and in particular, can protect MM cells against dexamethasone-induced apoptosis. It was shown in the present invention that PTK787 can overcome this protective effect of IL-6.

Hence, the present invention pertains also to a combination comprising a c-kit inhibitor, preferably a compound of formula I as defined above, and a compound effecting apoptosis of myeloma cells, in which the active ingredients are present in each case in free form or in the form of a pharmaceutically acceptable salt and optionally at least one pharmaceutically acceptable carrier, for simultaneous, separate or sequential use, especially for use in a method of treating myeloma. Preferably, in such combination the compound effecting apoptosis of myeloma cells is dexamethasone.

### SHORT DESCRIPTION OF THE FIGURES

FIG. 1: Effect of PTK787 on migration of MM.1S cells.

MM.1S cells are exposed to PTK787 at concentrations 0, 1, 5 and 10 μM for 4 h and then added to the upper chamber of a transwell system. To the lower wells are added either VEGF (□, 10 ng/ml) or CD40 (Δ, 10 ng/ml), and cells are allowed to migrate to the lower chamber over a 5 h period. A lower chamber without any VEGF or CD40 (∘) serves as a control. Migrated cells are counted on a Beckman Coulter counter, and a migration index is obtained as described in Example 2, below. Experiments are performed in triplicate, and standard deviation bars are shown.

FIGS. 2 to 4. Dose-related effect of PTK787 on proliferation of MM cell lines and patient tumor cells. MM.1S, MM.1R, RPMI 8226, patient PCL cells, patient MM cells and HUVE cells were incubated in the presence of 0 (□), 1 (▪), 5 ( [symbol] ), 10 ( [symbol] ) and 20 ( [symbol] ) μM of PTK787. In addition, HUVE cells are also exposed to 0.01 ( [symbol] ), and 0.1 ( [symbol] ) μM of drug. Proliferation is measured as a percentage of 3H-dT uptake relative to standard. The experiments are performed in triplicate, and standard deviation bars are shown.

A combination comprising a c-kit inhibitor and a compound effecting apoptosis of myeloma cells, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATION OF THE INVENTION can be a combined preparation or a pharmaceutical composition.

The term “a combined preparation”, as used herein defines especially a “kit of parts” in the sense that the active ingredients as defined above can be dosed independently or by use of different fixed combinations with distinguished amounts of the ingredients, i.e., simultaneously or at different time points. The parts of the kit can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the active ingredients. The ratio of the total amounts of the active ingredient 1 to the active ingredient 2 to be administered in the combined preparation can be varied, e.g., in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the first and second active ingredient, in particular a synergism, e.g. a more than additive effect, additional advantageous effects, less side effects, a combined therapeutical effect in a non-effective dosage of one or both of the first and second active ingredient, and especially a strong synergism the first and second active ingredient.

Additionally, the present invention provides a method of treating myeloma comprising administering a COMBINATION OF THE INVENTION in an amount which is jointly therapeutically effective against myeloma to a warm-blooded animal in need thereof.

The person skilled in the pertinent art is fully enabled to select relevant test models to prove the hereinbefore and hereinafter mentioned beneficial effects on myeloma of a compound inhibiting the c-kit activity or of a COMBINATION OF THE INVENTION. The pharmacological activity of a compound inhibiting the c-kit activity or a COMBINATION OF THE INVENTION may, for example, be demonstrated in a suitable clinical study or by means of the Examples described below. Suitable clinical studies are, for example, open label non-randomized, dose escalation studies in patients with advanced myeloma. Such studies prove in particular the synergism observed with the COMBINATIONS OF THE INVENTION. The beneficial effects on myeloma can be determined directly through the results of such studies or by changes in the study design which are known as such to a person skilled in the art. For example, one combination partner can be administered with a fixed dose and the dose of a second combination partner is escalated until the Maximum Tolerated Dosage (MTD) is reached. Alternatively, a placebo-controlled, double blind study can be conducted in order to prove the benefits of the COMBINATION OF THE INVENTION mentioned herein.

### EXAMPLES

### General

RPMI 8226 and U266 human MM cell lines are obtained from the American Type Culture Collection (ATCC) of Rockville, Md. Patient derived MM cells are purified from patient BM samples, as described by Y. T. Tai, G. Teoh, Y. Shima, et al in J. Immunol. Methods 235:11, 2000. All human MM cell lines are cultured in RPMI-1640 media (Sigma Chemical, St. Louis, Mo.), containing 10% fetal bovine serum (FBS), 2 mmol/L L-glutamine (L-glut, GIBCO, Grand Island, N.Y.), 100 U/mL penicillin and 100 mg/mL streptomycin (P/S, GIBCO). MM patient cells are ≧95% CD38+, CD45RA−. Bone marrow stromal cells (BMSCs) are prepared from aspirates of MM patients as well as healthy donors as described by D. Gupta, S. Treon, Y. Shima, et al in Leukemia, 2001 and S. Gartner and H. S. Kaplan in Proc. Natl. Acad. Sci. USA 77:4756, 1980. Cells are cultured in ISCOVE's modified Dulbecco media containing 20% FBS, 2 mmol/L L-glut, and 100 ug/mL P/S. Human umbilical vein endothelial cells (HUVEC P168) are purchased from Clonetics, Biowhittaker, and maintained in EGM-2MV media (Clonetics, Biowhittaker). PTK787 is dissolved in dimethyl sulfoxide (DMSO; Sigma) and stored as a 100 mM stock solution at −20° C. until used. For all assays, the compound is diluted in culture medium to concentrations ranging from 0.01 to 100 M. The concentration of DMSO is diluted to 0.1% for all assays.

Cytokine levels are measured in supernatants from the co-culture system described above. VEGF and IL-6 concentrations are measured using commercially available ELISA kits (R&D Systems). Statistical significance of differences is determined using unpaired Student's t-test. The minimal level of significance is considered as p<0.05.

### Cell Protein Lysates, Immunoprecipitation and Western Blot Analysis

MM cells are starved for 12 h in RPMI with 2% FBS, and then incubated for 1 h in RPMI-1640 without FBS in the presence PTK787 or DMSO control. These cells are subsequently stimulated with 100 nM VEGF₁₆₅ as described by K. Podar, Y. T. Tai, et al in Blood 98:428, 2001. Cells are then lysed in RIPA buffer containing 1 mM PMSF, 1 mM Sodium vanadate, and a protease inhibitor cocktail (Boehringer Mannheim). Lysates are either analyzed directly on a sodium dodecyl sulfate-polyacrylamide gel (SDS-PAGE gel) or incubated overnight with an antibody (Ab) against Flt-1, as well as protein G plus-Agarose (both from Santa Cruz Biotechnology, Calif.). Whole cell lysates (30 μg per lane) or immunoprecipitates are analyzed on an 8 to 10% SDS-PAGE get; transferred onto Hybond C Super paper (Amersham, Arlington Heights, Ill.); then probed with a murine MoAb against phospho-ERK, a murine MoAb against phospho-tyrosine residues, or Abs against Flt-1 or ERK2 (Santa Cruz); and detected using an HRP-conjugate anti-murine or anti-rabbit Ab (both from Santa Cruz) and enhanced chemiluminescence (ECL) substrate solution (Amersham).

### Proliferation and Cell Viability Assays

MM cells are first starved for 12 h in RPMI-1640 media containing 2% fetal bovine serum, and then plated into 96-well microtiter plates (Costar, Cambridge, Mass.), in the presence of drug or DMSO control. Experiments are also performed in the presence or absence of VEGF₁₆₅ (R and D Systems). Proliferation is measured by the incorporation of [³H]-thymidine (NEN Products, Boston, Mass.). Specifically, cells are pulsed with [³H]-thymidine (0.5 μCi/well) for the last 6 h of 48 h cultures, harvested onto glass filters with an automatic cell harvester (Cambridge Technology, Cambridge, Mass.), and counted using a LKB Betaplate scintillation counter (Wallac, Gaithersburg, Md.). Measurement of cell viability is performed colorimetrically by MTS assay, utilizing the CellTiter96 AQᵤₑₒᵤₛ One Solution Reagent (Promega, Madison, Wis.). Cells are exposed to the MTS for the last 2 h of 48 h cultures, and absorbance is measured using an ELISA plate reader (Molecular Devices Corp., Sunnyvale, Calif.) at OD of 570 nm.

### Cell Cycle Analysis

MM cells (1×10⁶ cells) are cultured in the presence of drug or DMSO control for 24, 48 and 72 h. Cells are then washed with phosphate buffered saline (PBS), fixed with 70% ethanol, and treated with RNAse (Sigma). Cells are next stained with propidium iodide (PI, 5 μg/mL), and the cell cycle profile is determined using the M software on an Epics flow cytometer (Coulter Immunology, Hialeah, Fla.).

### Example 1

### Expression of Flt-1, KDR, Flt-4 and c-Kit in MM Cells

For the detection of VEGF Receptors (Flt-1, Flt-4 and KDR), and c-kit by RT-PCR, total RNA is extracted from cell lines using the RNeasy Mini-kit (Qiagen, Valencia, Calif.). RT-PCR is performed in a thermal cycler (MJ Research, Watertown, Mass.) using 5 μg of total RNA, and 50 pM each of forward and reverse primers. RNA is amplified over 30 cycles using Superscript One-Step RT-PCR with Platinum Taq (Life Technologies, Gaithersburg, Md.). Primers to detect Flt-1, KDR, and Flt-4 are used as described by R. Masood, J. Cai, et al in Proc. Natl. Acad. Sci. USA 94:979, 1997; S. Dias, K. Hattori, et al, in J. Clin. Invest. 106:511, 2000; and E. Fournier, P. Dubreuil, et al in Oncogene 11:921, 1995, respectively. Expression of GADPH is used as a control to measure integrity of the RNA samples. To ensure that RNA samples are not contaminated with DNA, purified RNA is incubated with the appropriate primers and Taq polymerase, without reverse transcriptase.

Analysis of the PCR products reveals that Flt-1 is expressed in MM cells lines MM.1R, MM.1S, RPMI8226 and U266, as well as in tumor cells derived from a patient with plasma cell leukemia (PCL). Expression of c-Kit mRNA can be detected in all MM cell lines. By contrast, KDR is not expressed in any of the MM cell lines, and Flt-4 is expressed only in MM.1S and MM.1R cells.

### Example 2

### Transwell Cell Migration Assay

The migration assay is performed in a modified Boyden chamber system; using a 24 well plate with 8 μm pore size inserts. Prior to the assay, the upper and lower chambers are pre-coated with fibronectin (10 μg/ml). MM.1S cells are starved in 2% RPMI media for 6 hours prior to assay, then treated with drug or DMSO control for 4 h. MM cells (2×10⁶ cells/ml) are then placed into the upper chamber of the transwell system. To the lower chamber is added RPMI (1% FBS), 0 or 10 ng/ml of VEGF₁₆₅, or an activating MoAb to CD40 (10 ng/ml). The plates are then incubated at 37° C. for 6 h, and then cells in the lower chamber are harvested. The number of live migrated cells is gated and measured using a Beckman Coulter counter. A migration index is calculated to compare migration of cells relative to control. The migration index is defined herein as the percentage of live migrated cells in the sample (with or without drug, and ±VEGF), divided by the percentage of live migrated cells in the control (no drug, no VEGF).

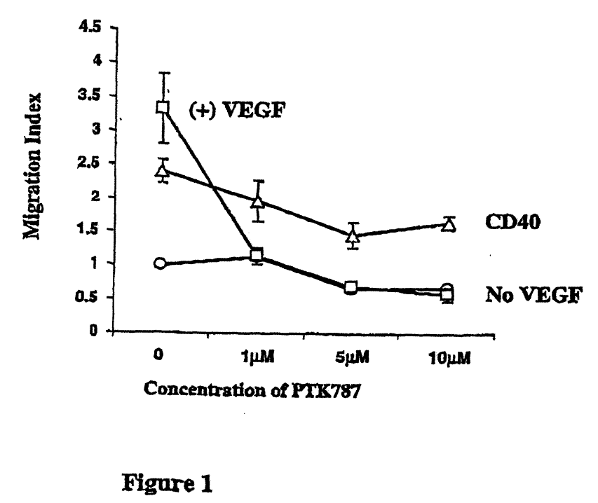

It is observed that VEGF increases migration of MM.1S cells by 3.4-fold (p=0.001), and this response is reduced by 1 μM PTK787 (p=0.002) (FIG. 1). Higher concentrations of PTK787 (5 and 10 μM) further reduces both baseline, as well as VEGF-induced, migration of MM.1S cells. In contrast, PTK787 does not significantly inhibit migration of MM.1S cells induced by CD40.

### Example 3

### Proliferation of MM Cells in an Adhesion System

BMSCs (1×10⁴ cells/well) are plated into 96-well microtiter plates and incubated at 37° C. for 24 h in ISCOVE's media (20% FBS). MM cells are then added to the BMSC-containing wells (5×10⁴ cells/well), in the presence of drug or DMSO control. When MM.1S cells are used, both BMSCs and MM cells are starved for 12 h in RPMI-1640 media containing 2% FBS. When patient PCL cells are used, the co-cultures are performed in RPMI media containing 10% FBS. BMSCs and MM cells are also cultured separately to serve as controls. After 48 h, proliferation and cell viability are analyzed as described above. To ensure that al cells are collected for the proliferation assay, 10× Trypsin (Sigma) is added to each well 10 minutes prior to harvesting.

### Example 4

### Proliferation of MM Cells in a Modified Boyden Chamber Transwell System

Proliferation is measured in a modified Boyden chamber transwell system, using 24-well plates with a 0.4 mm pore size inserts (Costar). BMSCs (4×10⁴ cells/well) are plated in the lower chamber, starved, and incubated in drug as described above. MM cells (20×10⁴ cells/ml) are then placed in the upper chamber (insert), and [³H]-thymidine uptake in the individual chambers is measured at 48 h as described above.

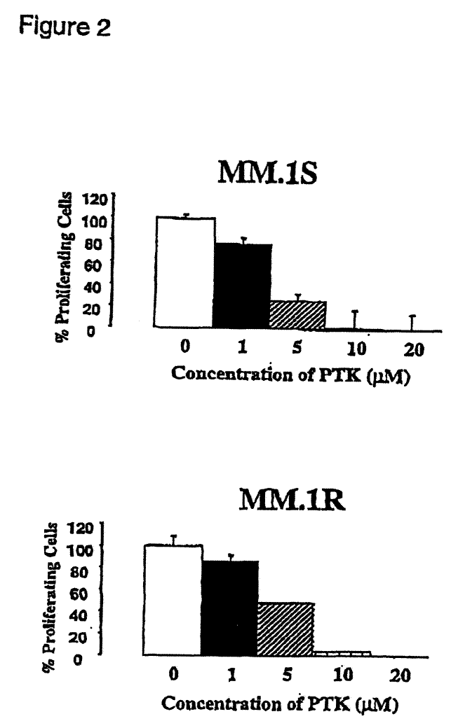

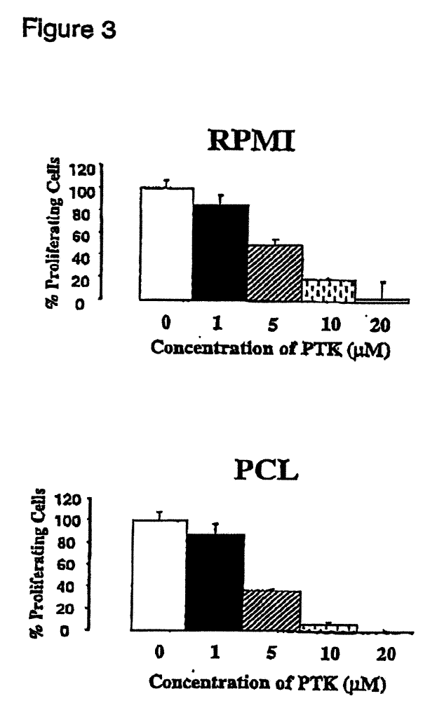

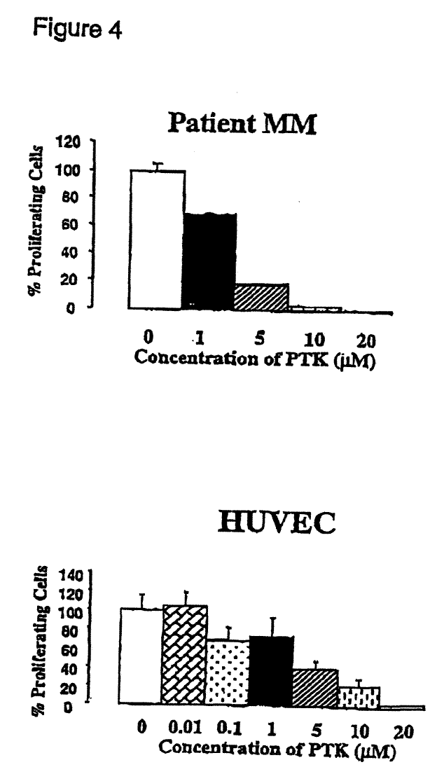

As shown in FIGS. 2 to 4, PTK787 decreases, in a dose dependent manner proliferation of MM.1S, Dex-resistant MM.1R, RPMI, patient plasma cell leukemia (PCL) and patient MM cells. VEGF (50 ng/ml) stimulates modest, but not significant, increases in MM growth. The IC₅₀ of PTK787 on most MM cell lines tested is 1 and 5 μM. The IC₅₀ of PTK787 on HUVE cells is between 0.1 and 1 μM.

### Example 5

### Effect of PTK787 on Proliferation MM.1S Cells

MM.1 S cells are placed in the upper chamber of a transwell co-culture system in order to preclude direct contact between MM cells and BMSCs, but nonetheless allow for diffusion of humoral factors. Despite the lack of contact between the two cell types, uptake of [³H]-dT by MM.1S cells incubated with BMSCs is increased by 2.2-fold (p<0.0001) at 48 h. By contrast, the BMSCs in the co-culture system do not show a significant increase in [³H]-dT uptake. When PTK787 (5 μM) is used in this co-culture system, it reduces proliferation of MM.1S cells by 62% (p<0.001).

### Example 6

### Effect of PTK787 on Proliferation MM.1s Cells in the presence of Dexamethasone and IL-6

MM.1S cells are incubated for 24 h in 1% RPMI, in the presence or absence of dexamethasone (0.1 or 1 μM), PTK787 (5 μM), IL-6 (20 and 100 ng/ml) and VEGF (50 ng/ml). Dexamethasone and PTK787 inhibit proliferation of MM.1S cells at 24 h by 58% (p<0.001) and 35% (p<0.001), respectively and survival by 83% (p<0.001) and 46% (p<0.001), respectively. IL-6 increases MM.1S proliferation by 17%, compared to a DMSO control (p<0.002), and protects MM.1S cells against dexamethasone (16% inhibition with IL-6 versus 70.5% without IL-6, p<0.001). The addition of IL-6 to cells already exposed to PTK787 further inhibits MM.1S proliferation (64.7% vs. 39.3%, p<0.001), and increases cell death (54% vs. 65%, p<0.01). Additionally, the combination of PTK787 and dexamethasone totally abrogates the ability of IL-6 to promote growth, as well as protect against dexamethasone-induced apoptosis in MM.1S cells. These effects are noted in dexamethasone concentrations from 0.1 to 1 μM. Even high (100 ng/ml) concentrations of IL-6 do not rescue MM.1S cells from growth inhibition by the combination of dexamethasone and PTK787.

### Example 7

### Correlation of Increases in Proliferation of MM Cells and BMSCs with Increases in IL-6 Concentration

Supernatants obtained from a co-culture of MM.1S cells with BMSCs are isolated at 48 h, and IL-6 levels are measured using ELISA assay. IL-6 levels increase (7.9-fold, p<0.001) in the co-cultures of MM cells and BMSCs, compared to cultures of BMSCs alone. IL-6 secretion from MM.1 S cells is below detectable limits. Addition of PTK787 to the co-culture system significantly decreases (by 70%) IL-6 concentrations, correlating with decreased proliferation of adherent MM cells. Viability of BMSCs, analyzed by both trypan blue exclusion and MTS assay, remains high (95%) suggesting that PTK787 reduces secretion of IL-6 without killing BMSCs.

### Example 8

### Effects of PTK787 on the Proliferation of MM Cells in a Co-Culture System

Co-culturing of MM.1S cells or patient PCL cells with BMSCs increases growth of tumor cells by 1.8 to 2.8-fold, compared to the combination of BMSCs and MM cells alone. Addition of PTK787 (5 μM) to this co-culture system decreases proliferation of both MM.1S cells and patient PCL cells by 75% (p<0.0001), when compared to controls. These effects are observed whether the BMSCs are derived from a normal donor or patient BM.

It is shown in the Examples that all MM cells express c-Kit. The ubiquitous expression of c-Kit by MM cells demonstrates the potential use of c-Kit tyrosine kinase inhibitors such as the compounds of formula I or the compound known as STI571B (Gleevec®, N-{5-[4-(4-methyl-piperazino-methyl)-benzoylamido]-2-methylphenyl}-(3-pyridyl)-2-pyrimidine-amine monomesylate, disclosed in WO 99/03854, especially in Examples 4 and 6) in inhibiting MM cell growth and, hence, in the treatment of myeloma.

Furthermore, the Examples demonstrate that

(1) PTK787 (1 to 5 M) inhibits proliferation in MM cell lines. PTK787 abrogates proliferation both in the presence or absence of VEGF;
(2) VEGF-induced migration of MM.1 S cells is effectively reduced to baseline levels at 1 μM concentration of PTK787;
(3) dexamethasone and PTK787 independently inhibit proliferation of MM.1S cell, and the inhibitory effects are at least additive;
(4) importantly, PTK787 overcomes the stimulatory effects of IL-6 on MM cell growth and is able to overcome IL-6-induced resistance to dexamethasone-induced apoptosis, even at high (100 ng/ml) IL-6 concentrations;
(5) PTK787 abrogates the increase in proliferation of MM cells co-cultured with BMSCs. The fact that PTK787 can inhibit MM cell proliferation in a co-culture system suggests that the drug can overcome protection conferred by adherence of MM cells to BMSCs.

In summary, the Examples show that PTK787 at clinically achievable concentrations has both direct effects on MM cells, as well as on MM cell interactions with BMSCs.

The results obtained with PTK787 in the models disclosed herein as well as further tests and test data are described in Cancer Research 2002, 62, 5019-5026, which publication is included herein by reference.

It is one objective of this invention to provide a pharmaceutical composition comprising a quantity, which is jointly therapeutically effective against myeloma comprising the COMBINATION OF THE INVENTION. In this composition, the combination partners can be administered together, one after the other or separately in one combined unit dosage form or in two separate unit dosage forms. The unit dosage form may also be a fixed combination.

The pharmaceutical compositions for separate administration of the combination partners and for the administration in a fixed combination, i.e. a single galenical composition comprising at least two combination partners, according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of treatment of myeloma according to the present invention may comprise (i) administration of a combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of the compounds used for inhibiting the c-kit activity and of the combination partners employed in the COMBINATION OF THE INVENTION may vary depending on the particular compound or pharmaceutical composition employed, the mode of administration, the type of the myeloma being treated, the severity of the myeloma being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of compounds inhibiting the c-kit activity or of the single active ingredients of the COMBINATION OF THE INVENTION required to prevent, counter or arrest the progress of the condition. Optimal precision in achieving concentration of the active ingredients within the range that yields efficacy without toxicity requires a regimen based on the kinetics of the active ingredients' availability to target sites.

N-{5-[4-(4-methyl-piperazino-methyl)-benzoylamido]-2-methylphenyl}-4-(3-pyridyl)-2-pyrimidine-amine monomesylate is preferably administered to a human in a dosage in the range of about 5 to 850 mg/day, more preferably 25 to 600 mg/day and most preferably 100 to 300 mg/day. Unless stated otherwise herein, the compound is preferably administered between one and four times per day.

If the warm-blooded animal is a adult human, the dosage of a compound of formula I, especially PTK787, is preferably in the range of about 100 to 1500, more preferably about 250 to 1250, and most preferably 500 to 1000, mg/day.

Moreover, the present invention provides a commercial package comprising as active ingredients the COMBINATION OF THE INVENTION, together with instructions for simultaneous, separate or sequential use thereof in the treatment of myeloma.

The present invention also provides the use of a compound of formula I as defined herein and the use of a COMBINATION OF THE INVENTION for the preparation of a medicament for the treatment of myeloma.

What is claimed:

1. A method of treating myeloma comprising administering to a warm-blooded animal in need thereof a therapeutically effective amount of a compound of formula I 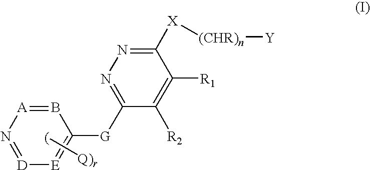
wherein
r is 0 to 2,
n is 0 to 2,
m is 0 to 4,
R₁ and R₂ (i) are lower alkyl or
(ii) together form a bridge in subformula I* 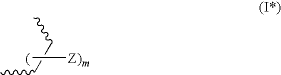
the bridge being achieved via the two terminal carbon atoms, or
(iii) together form a bridge in subformula I** 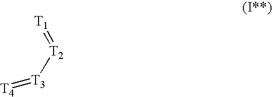
wherein one or two of the ring members T₁, T₂, T₃ and T₄ are nitrogen, and the others are in each case CH, and the bridge is achieved via T₁ and T₄;
A, B, D, and E are, independently of one another, N or CH, with the stipulation that not more than 2 of these radicals are N;
G is lower alkylene, lower alkylene substituted by acyloxy or hydroxy, —CH₂—O—, —CH₂—S—, —CH₂—NH—, oxa (—O—), thia (—S—), or imino (—NH—);
Q is lower alkyl;
R is H or lower alkyl;
X is imino, oxa, or thia;
Y is unsubstituted or substituted aryl, pyridyl, or unsubstituted or substituted cycloalkyl; and
Z is amino, mono- or disubstituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfonyl, phenyl-lower alkylsulfinyl or alkylphenylsulfinyl, substituents Z being the same or different from one another if more than 1 radical Z is present;
and wherein the bonds characterized, if present, by a wavy line are either single or double bonds;
or an N-oxide of the defined compound, wherein 1 or more N atoms carry an oxygen atom,
or the salt of such compound having at least one salt-forming group.

2. A combination comprising PTK787 and a compound effecting apoptosis of myeloma cells, in which the active ingredients are present in each case in free form or in the form of a pharmaceutically acceptable salt and optionally at least one pharmaceutically acceptable carrier, for simultaneous, separate or sequential use, wherein the compound effecting apoptosis of myeloma cells is dexamethasone.

3. Combination according to claim 2 for simultaneous, separate or sequential use in the treatment of myeloma.

4. A method of treating myeloma comprising administering a combination as defined in claim 2 in an amount which is jointly therapeutically effective against myeloma to a warm-blooded animal in need thereof.

5. A pharmaceutical composition comprising a quantity, which is jointly therapeutically effective against myeloma, of a combination according to claim 2 and at least one pharmaceutically acceptable carrier.
